(19) United States
(12) Patent Application Publication
(10) Pub. No.: US 2021/0186460 A1
(43) Pub. Date: Jun. 24, 2021

(54) METHOD OF SPATIALLY LOCATING POINTS OF INTEREST DURING A SURGICAL PROCEDURE

(71) Applicant: Covidien LP, Mansfield, MA (US)

(72) Inventor: Dwight Meglan, Westwood, MA (US)

(21) Appl. No.: 16/636,053

(22) PCT Filed: Aug. 13, 2018
(86) PCT No.: PCT/US2018/046419

§ 371 Date: Feb. 3, 2020

Related U.S. Application Data
(60) Provisional application No. 62/546,054, filed on Aug. 16, 2017.

(51) Int. Cl. A61B 8/00; A61B 8/12; A61B 90/00

(52) U.S. Cl. CPC A61B 8/4245; A61B 8/12; A61B 8/464; A61B 2090/3782; A61B 90/361; A61B 90/37; A61B 8/463

(57) ABSTRACT

A method of visualizing a surgical site includes scanning a surgical site with an ultrasound system, marking a first area or point of interest within a cross-sectional view of the surgical site with a first tag, viewing the surgical site with a camera, and showing an image of the surgical site captured by the camera on a second display. The second display displays a first indicia representative of the first tag on the image of the surgical site captured by the camera.

### BACKGROUND

During a minimally invasive surgery (MIS) an intraoperative ultrasound probe can be used to provide two-dimensional (2D) cross-sectional views of a surgical site. During MIS, a clinician typically holds the ultrasound probe, either with a surgical grasper tool or with the ultrasound probe being part of its own dependent tool shaft. The ultrasound probe is placed in contact with a tissue region of interest and moved about so that the 2D cross-sectional view image of the surgical site is seen on an ultrasound display. The ultrasound display is typically distinct from an endoscope display which is showing images captured from an endoscope being used to directly observe the surgical site. The endoscope display may be used to direct manipulation of the ultrasound probe.

The 2D cross-sectional views can reveal information about the state of the structures below the tissue surface at/or adjacent the surgical site. Typically, a clinician manipulates the ultrasound probe and mentally notes the structures at/or adjacent the surgical site. After the clinician removes the ultrasound probe to begin or continue the surgical procedure, the clinician must remember location of the structures at/or adjacent the surgical site. If during the surgical procedure the clinician requires a reminder of the 2D cross-sectional views, the surgical procedure is paused and the ultrasound probe is reactivated to reacquire 2D cross-sectional views and refresh a clinician's memory. This pausing of the surgical procedure can cause a disruption in the flow of the surgical procedure. This disruption in flow of the surgical procedure may encourage a clinician not to pause the surgical procedure to reacquire the 2D cross-sectional views with the ultrasound probe. By not pausing during a surgical procedure to reacquire the 2D cross-sectional views, quality of decision making during a surgical procedure may be reduced.

There is a need to allow a clinician to view ultrasound images during a surgical procedure at points of interest during the surgical procedure. By identifying points of interest during a surgical procedure, surgical decision making can be improved.

### SUMMARY

In an aspect of the present disclosure, a method of visualizing a surgical site includes scanning a surgical site with an ultrasound system, marking a first area or point of interest within cross-sectional views of the surgical site with a first tag, and viewing the surgical site with a camera on a second display. The second display displaying a first indicia representative of the first tag.

In aspects, scanning the surgical site with the ultrasound system includes inserting an ultrasound probe into a body cavity of a patient. Displaying the first indicia representative of the first tag may include displaying information relevant to the first area or point of interest on the second display. The method may include toggling the first indicia to display information relevant to the first area or point of interest on the second display.

In some aspects, viewing the surgical site with the camera on the second display incudes a control unit locating the first tag within images captured by the first camera. Locating the first tag within images captured by the camera may include determining a depth of the first tag within the surgical site from multiple images captured by the camera. Locating the first tag within images captured by the camera may include using pixel-based identification of images from the camera to determine the location of the first tag within the images captured by the camera.

In particular aspects, the method includes freezing the first display such that a particular cross-sectional view of the surgical site is viewable on the first display. Viewing the surgical site with the camera on the second display may include removing distortion from the images of the surgical site captured with the camera before displaying the images of the surgical site on the second display.

In certain aspects, the method includes marking a second area or point of interest within the cross-sectional views of the surgical site with a second tag and viewing a second indicia representative of the second tag on the second display. Viewing the second indicia representative of the second tag includes displaying information relevant to the second area or point of interest on the second display. The method may include toggling the second indicia to display information relevant to the second area or point of interest on the second display. The method may also include toggling the first indicia to display information relevant to the first area or point of interest on the second display independent of toggling the second indicia.

In another aspect of the present disclosure, a surgical system includes an ultrasound system, an endoscopic system, and a processing unit. The ultrasound system includes an ultrasound probe and an ultrasound display. The ultrasound probe is configured to capture cross-sectional views of a surgical site. The ultrasound display is configured to display the cross-sectional views of the surgical site captured by the ultrasound probe. The endoscopic system includes an endoscope and an endoscope display. The endoscope has a camera that is configured to capture images of the surgical site. The endoscope display is configured to display the images of the surgical site captured by the camera. The processing unit is configured to receive a location of a first area or point of interest within a cross-sectional view of the surgical site and to display a first indicia representative of the first area or point of interest on the second display.

In aspects, the ultrasound display is a touchscreen display that is configured to receive a tag that is indicative of the location of the first area or point of interest within the cross-sectional view of the surgical site. The processing unit may be configured to remove distortion from images of the surgical site captured with the camera before displaying the images of the surgical site on the second display. The processing unit may be configured to locate the first area or point of interest within images captured by the camera using pixel-based identification of images from the camera.

Further, to the extent consistent, any of the aspects described herein may be used in conjunction with any or all of the other aspects described herein.

### BRIEF DESCRIPTION OF THE DRAWINGS

Various aspects of the present disclosure are described hereinbelow with reference to the drawings, which are incorporated in and constitute a part of this specification, wherein:

FIG. 1 is a perspective view of an ultrasound system in accordance with the present disclosure including an ultrasound probe, a positional field generator, a processing unit, an ultrasound display, and an endoscope display;

FIG. 2 is a cut-away of the detail area shown in FIG. 1 illustrating the ultrasound probe shown in FIG. 1 and an endoscope within a body cavity of a patient;

FIG. 3 is view of the ultrasound display of FIG. 1 illustrating a two-dimensional cross-sectional image of a surgical site; and

FIG. 4 is view of the endoscope display of FIG. 1 illustrating an image of the surgical site and a distal portion of a surgical instrument within the surgical site.

### DETAILED DESCRIPTION

Embodiments of the present disclosure are now described in detail with reference to the drawings in which like reference numerals designate identical or corresponding elements in each of the several views. As used herein, the term “clinician” refers to a doctor, a nurse, or any other care provider and may include support personnel. Throughout this description, the term “proximal” refers to the portion of the device or component thereof that is closest to the clinician and the term “distal” refers to the portion of the device or component thereof that is farthest from the clinician.

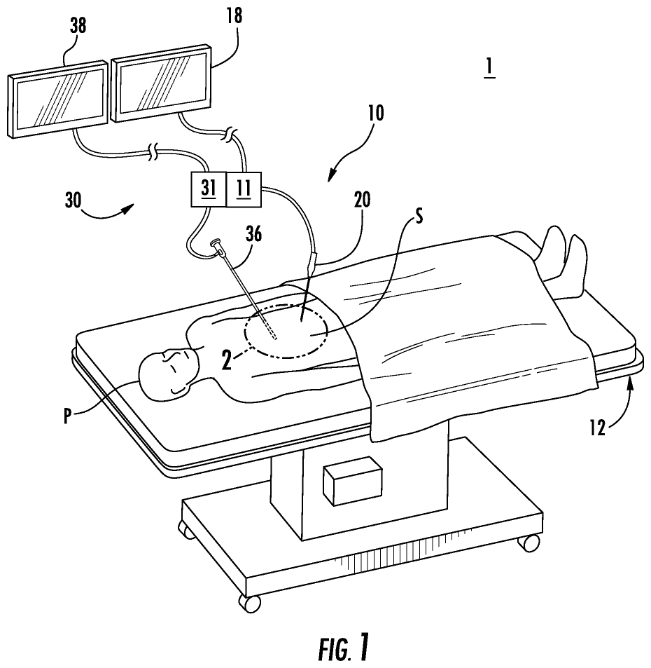

Referring now to FIG. 1, a surgical system 1 provided in accordance with the present disclosure includes an ultrasound imaging system 10 and an endoscopic system 30. The ultrasound imaging system 10 includes a processing unit 11, an ultrasound display 18, and an ultrasonic probe 20.

The ultrasound imaging system 10 is configured to provide 2D cross-sectional views or 2D image slices of a region of interest within a body cavity of a patient “P” on the ultrasound display 18. A clinician may interact with the ultrasound imaging system 10 and an endoscope 36, which may include a camera, to visualize surface and subsurface portions of a surgical site “S” of the patient “P” during a surgical procedure as detailed below.

The ultrasound probe 20 is configured to generate 2D cross-sectional views of the surgical site “S” from a surface of a body cavity of the patient “P” and/or may be inserted through an opening, either a natural opening or an incision, to be within the body cavity adjacent the surgical site “S”. The processing unit 11 receives 2D cross-sectional views of the surgical site “S” and transmits a representation of the 2D cross-sectional views on the ultrasound display 18.

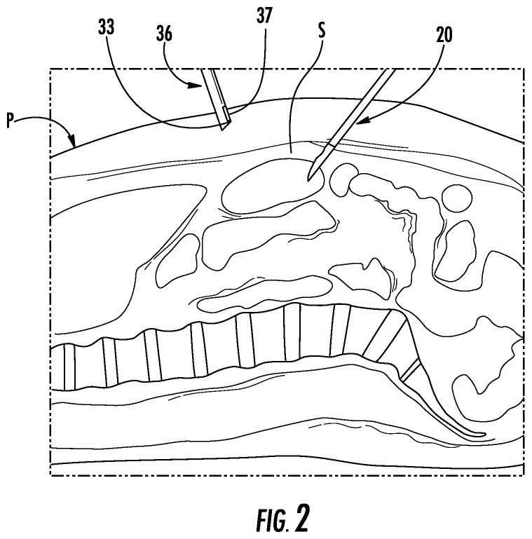

The endoscopic system 30 includes a control unit 31, an endoscope 36, and an endoscope display 38. With additional reference to FIG. 2, the endoscope 36 may include a camera 33 and a sensor 37 which are each disposed on or in a distal portion of the endoscope 36. The camera 33 is configured to capture images of the surgical site “S” which are displayed on the endoscope display 38. The control unit 31 is in communication with the camera 33 and is configured to transmit images captured by the camera 33 to the endoscope display 38. The control unit 31 is in communication with the processing unit 11 and may be integrated with the processing unit 11.

Referring to FIGS. 1-4, the use of the ultrasound system 10 and the endoscopic system 30, to image the surgical site “S”, is described in accordance with the present disclosure. Initially, the ultrasound probe 20 is positioned adjacent the surgical site “5”, either within or outside of a body cavity of the patient, to capture 2D cross-sectional views of the surgical site “S”. The ultrasound probe 20 is manipulated to provide 2D cross-sectional views of the areas or points of interest at or adjacent the surgical site “S”. It will be appreciated that the entire surgical site “S” is scanned while the ultrasound probe 20 is within the view of the camera 33 of the endoscope 36 such that the position of ultrasound probe 20 can be associated with the 2D cross-sectional views of the surgical site “S” as the 2D cross-sectional views are acquired. While the surgical site “S” is scanned, the processing unit 11 and/or the control unit 31 record the 2D cross-sectional views and associate the 2D cross-sectional views with the position of the ultrasound probe 20 within the surgical site “S” at the time each 2D cross-sectional view was acquired.

When the endoscope 36 views the surgical site “S”, the camera 33 of the endoscope 36 captures real-time images of the surgical site “S” for viewing on the endoscope display 38. After the surgical site “S” is scanned with the ultrasound probe 20, other surgical instruments, e.g., a surgical instrument in the form of a grasper or retractor 46, may be inserted through the same or a different opening from the endoscope 36 to access the surgical site “S” to perform a surgical procedure at the surgical site “S”.

As detailed below, the 2D cross-sectional views of the surgical site “S” recorded during the scan of the surgical site “S” are available for view by the clinician during the surgical procedure. As the camera 33 captures real-time images, the images are displayed on the endoscope display 38. The clinician may select an area or point of interest of the surgical site “S” to review on the endoscope display 38. When the an area or point of interest is selected on the endoscope display 38, the control unit 31 determines the position of the area or point of interest within the surgical site “S” and sends a signal to the processing unit 11. The processing unit 11 receives the signal from the control unit 31 and displays a recorded 2D cross-sectional view taken when the ultrasound probe 20 was position at/or near the area or point of interest during the scan of the surgical site “S”. The recorded 2D cross-sectional view can be a fixed image or can be a video clip of the area or point of interest.

When the recorded 2D cross-sectional view is a video clip of the area or point of interest the video clip may have a duration of about 1 second to about 10 seconds. The duration of the video clip may be preset or may be selected by the clinician before or during a surgical procedure. It is envisioned that the video clip may be looped such that it continually repeats.

To indicate the area or point of interest on the endoscope display 38, the clinician may electronically or visually “mark” or “tag” the area or point of interest in the image on the endoscope display 38. To electronically or visually mark the area or point of interest in the image on the endoscope display 38, the clinician may use any known means including, but not limited to, touching the display with a finger or stylus; using a mouse, track pad, or similar pointing device to move an indicator on the endoscope display 38; using a voice recognition system; using an eye tracking system; typing on a keyboard; and/or a combination thereof.

To determine the position of the area or point of interest within the surgical site “S”, the control unit 31 processes the real-time images from the camera 33. The control unit 31 may remove distortion from the real-time images to improve accuracy of determining the position of the area or point of interest. It is envisioned that the control unit 31 may utilize a pixel-based identification of the real-time images from the camera 33 to identify the location of the area or point of interest within the real-time images from the camera 33. Additionally or alternatively, the location of the area or point of interest may be estimated from multiple real-time images from the camera 33. Specifically, multiple camera images captured during movement of the endoscope 36 about the surgical site “S” can be used to estimate a depth of an area or point of interest within the surgical site “S”.

In embodiments, a stereoendoscope can be used to determine a depth of structures within the surgical site “S” based on the depth imaging capability of the stereoendoscope. The depth of the structures can be used to more accurately estimate the location of the area or point of interest in the images from the camera 33.

With the location of the area or point of interest of the surgical site “S” determined, the processing unit 11 displays a 2D cross-sectional view, recorded during the scan of the surgical site “S” detailed above, that is associated with the identified location of the area or point of interest. The clinician can observe the 2D cross-sectional view to visualize subsurface structures at the area or point of interest. By visualizing the subsurface structures at the area or point of interest, the clinician's situational awareness of the area or point of interest is improved without the need for rescanning the area or point of interest with the ultrasound probe 20.

Additionally or alternatively, during a surgical procedure, a clinician may rescan an area or point of interest within the surgical site “S” with the ultrasound probe 20 to visualize a change effected by the surgical procedure. It is envisioned that the clinician may visualize the change on the ultrasound display 18 by comparing the real-time 2D cross-sectional views with the recorded 2D cross-sectional views at the area or point of interest. To visualize the changes on the ultrasound display 18, the clinician may overlay either the real-time or recorded 2D cross-sectional view with the other,

Before, during, or after viewing 2D cross-sectional views, the clinician may “tag” areas or points of interest within images on the endoscope display 38, as represented by tags 62, 64, 66 in FIG. 4. The tags 62-66 may include information about the area or point of interest which may not be apparent when the surgical site “S” is viewed with the endoscope 36, e.g., nerve, blood vessel, scar tissue, blood flow, etc., about the area or point of interest. It is contemplated that the clinician may freeze the image on the endoscope display 38 before, after, or during tagging of the area or point of interest. With the area or point of interest tagged on the endoscope display 38, the clinician may continue the surgical procedure. Similar to marking the area or point of interest, the clinician may use any known means to tag an area or point of interest on the endoscope display 38.

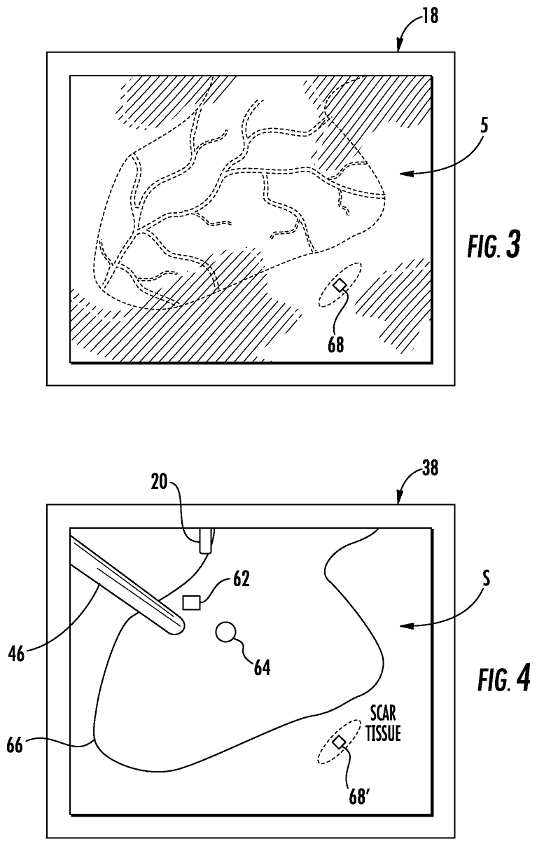

Additionally, while viewing the ultrasound display 18, the clinician may identify an area or point of interest at or adjacent the surgical site “S”. When the clinician identifies an area or point of interest on the display 18, the clinician may electronically or visually “mark” or “tag” the area or point of interest in the image on the display 18 as represented by tag 68 in FIG. 3. To electronically or visually tag the area or point of interest in the image on the display 18, the clinician may use any known means as detailed above. The tag 68 may include information about the area or point of interest which may not be apparent when the surgical site “S” is viewed with the endoscope 36, e.g., nerve, blood vessel, scar tissue, blood flow, etc., about the area or point of interest. It is contemplated that the clinician may freeze the image on the ultrasound display 18 before, after, or during tagging of the area or point of interest. With the area or point of interest tagged on the ultrasound display 18, the clinician may continue to scan the surgical site “S” with the ultrasound probe 20 and electronically or visually tag subsequent areas or points of interest on the display 18 indicative of areas or points of interest at or adjacent the surgical site “S”. With the area or point of interest tagged on the ultrasound display 18, the clinician may continue to scan the surgical site “S” with the ultrasound probe 20 and electronically or visually tag subsequent areas or points of interest on the ultrasound display 18 indicative of areas or points of interest at or adjacent the surgical site “S”.

When an area or point of interest is tagged on ultrasound display 18, e.g., tag 68, the location of the ultrasound probe 20 within the surgical site “S” is marked on the endoscope display 38 with a tag, e.g., tag 68′, to represent the tag on the ultrasound display 18.

Providing tags 62, 64, 66, 68′ with information of areas or points of interest at or adjacent a surgical site during a surgical procedure without requiring a clinician to pause a procedure may increase a clinician's situational awareness during a surgical procedure and/or may decrease a clinician's cognitive loading during a surgical procedure. Increasing a clinician's situational awareness and/or decreasing a clinician's cognitive loading may improve surgical outcomes for patients.

As shown, the tags 62, 64, 66, 68′ can be displayed in a variety of shapes including a sphere, a cube, a diamond, an exclamation point. The shape of the tags 62, 64, 66, 68′ may be indicative of the type of information pertinent to the associated tags 62, 64, 66, 68′. In addition, the tags 62, 64, 66, 68′ may have a color indicative of the information contained in the tag. For example, the tag 62 may be blue when the information of the tag is pertinent to a blood vessel or may be yellow when the information of the tag is pertinent to tissue.

It is contemplated that the tags 62, 64, 66, 68′ may be saved for subsequent surgical procedures. Before a surgical procedure on a patient, a clinician can load a profile of the patient into the processing unit 11 and/or the control unit 31 including tags from a previous procedure. As the camera 33 of the endoscope 36 captures real-time images, the control unit 31 identifies structures within the surgical site “S” to locate and place tags, e.g., tags 62, 64, 66, 68′ from previous surgical procedures. When similar structures are identified within the surgical site “S” the control unit 31 places a tag within the image on the endoscope display 38 to provide the clinician with additional information about and/or 2D cross-sectional views of the area or point of interest from the previous surgical procedure in a similar manner as detailed above.

As detailed above and with reference back to FIG. 1, the surgical system 1 includes an ultrasound display 18 and a separate endoscope display 38. However, the surgical system 1 can include a single monitor having a split-screen of multiple windows and/or panels with each of the ultrasound display 18 and the endoscope display 38 viewable in a respective one of the windows or panels on the monitor.

While several embodiments of the disclosure have been shown in the drawings, it is not intended that the disclosure be limited thereto, as it is intended that the disclosure be as broad in scope as the art will allow and that the specification be read likewise. Any combination of the above embodiments is also envisioned and is within the scope of the appended claims. Therefore, the above description should not be construed as limiting, but merely as exemplifications of particular embodiments. Those skilled in the art will envision other modifications within the scope of the claims appended hereto.

What is claimed:

1. A method of visualizing a surgical site, the method comprising:
scanning a surgical site with an ultrasound system including a first display showing a cross-sectional view of the surgical site including recording cross-sectional views of the surgical site, each of the recorded cross-sectional views associated with a position a probe of the ultrasound system within the surgical site when the respective cross-sectional view is recorded;
viewing the surgical site with a camera on a second display;
identifying a first area of interest on the second display such that a recorded cross-sectional view of the surgical sited associated with the first area of interest on the second display is displayed on the first display.

2. The method according to claim 1, wherein scanning the surgical site with the ultrasound system includes inserting an ultrasound probe into a body cavity of a patient.

3. The method according to claim 1, further comprising marking a second area of interest on the second display with a first tag including information relative to the second area of interest.

4. The method according to claim 3, further comprising toggling the first tag to display information relevant to the second area of interest on the second display.

5. The method according to claim 3, wherein marking the second area of interest includes identifying the second area of interest within the first area of interest.

6. The method according to claim 1, further comprising locating a first tag within images captured by the camera based on a position of a previous area of interest during a prior surgical procedure.

7. The method according to claim 6, wherein displaying the first tag representative of the previous area of interest includes displaying information relevant to the previous area of interest on the second display.

8. The method according to claim 7, further comprising toggling the first tag to display information relevant to the previous area of interest on the second display.

9. The method according to claim 6, wherein locating the first tag within images captured by the camera includes determining a depth of the first tag within the surgical site from multiple images captured by the camera.

10. The method according to claim 6, wherein locating the first tag within images captured by the camera includes using pixel-based identification of images from the camera to determine the location of the first tag within the images captured by the camera.

11. The method according to claim 1, wherein viewing the surgical site with the camera on the second display includes removing distortion from images of the surgical site captured with the camera before displaying the images of the surgical site on the second display.

12. The method according to claim 1, further comprising:
marking a third area of interest within a cross-sectional view of the surgical site on the first display with a second tag; and
viewing a third tag on the second display representative of the position of the probe of the ultrasound within images captured by the camera when the third area of interest was identified.

13. The method according to claim 12, wherein viewing the third tag representative of the second tag includes displaying information relevant to the third area of interest on the second display.

14. The method according to claim 13, further comprising toggling the third tag to display information relevant to the third area of interest on the second display.

15. The method according to claim 14, further comprising toggling the first tag to display information relevant to the first area of interest on the second display independent of toggling the third tag.

16. A surgical system comprising:
an ultrasound system including:
an ultrasound probe configured to capture a cross-sectional view of a surgical site; and
an ultrasound display configured to display the cross-sectional view of the surgical site captured by the ultrasound probe;
an endoscopic system including:
an endoscope having a camera configured to capture images of the surgical site;
an endoscope display configured to display the images of the surgical site captured by the camera; and
a processing unit configured to receive a location of a first area of interest within a captured image of the surgical site from the endoscope display and to display a cross-sectional view of the surgical site at the location on the endoscope display.

17. The surgical system according to claim 16, wherein the endoscope display is a touchscreen display configured to receive a tag indicative of the location of the first area of interest within the images of the surgical site.

18. The surgical system according to claim 16, wherein the processing unit is configured to remove distortion from images of the surgical site captured with the camera before displaying the images of the surgical site on the endoscope display.

19. The surgical system according to claim 16, wherein the processing unit is configured to locate a second area of interest within images captured by the camera using pixel-based identification of images from the camera, the second area of interest positioned based on a location of the ultrasound probe within the images of the surgical site when a second area of interest is identified on the ultrasound display.
